(19) United States
(12) Patent Application Publication
(10) Pub. No.: US 2025/0024928 A1
(43) Pub. Date: Jan. 23, 2025

(54) HAIRCARE APPLIANCE

(71) Applicant: Dyson Technology Limited, Malmesbury, Wiltshire (GB)

(72) Inventors: Nathan Haruna Waters IMHASLY, Bath (GB); Thomas Alexander CARLYLE, Chippenham (GB); Adam BRANN, Bristol (GB)

(21) Appl. No.: 18/709,057

(22) PCT Filed: Nov. 8, 2022
(86) PCT No.: PCT/GB2022/052814

§ 371 Date: May 10, 2024

(30) Foreign Application Priority Data
Nov. 10, 2021 (GB) 2116174.0

(51) Int. Cl. A45D 20/12; A45D 44/00

(52) U.S. Cl. CPC A45D 20/122; A45D 2044/007

(57) ABSTRACT

A haircare appliance has a main body housing an airflow generator for generating an airflow, an intermediate attachment for detachably connecting to the main body, and a hair treatment attachment for detachably connecting to the intermediate attachment. The intermediate attachment has an inlet for receiving the airflow from the main body and an outlet for emitting the airflow. The hair treatment attachment is configured to receive the airflow from the outlet of the intermediate attachment and emit the airflow towards hair to be treated in use. The intermediate attachment has a sensor module for sensing a property of at least one of hair to be treated and the hair treatment attachment.

### FIELD OF THE INVENTION

The present invention relates to a haircare appliance.

### BACKGROUND OF THE INVENTION

Haircare appliances are generally used to treat hair, for example to dry or style hair. In some haircare appliances, a number of attachments may be provided, with the attachments providing different drying or styling characteristics.

### SUMMARY OF THE INVENTION

The present invention provides a haircare appliance comprising a main body housing an airflow generator for generating an airflow; an intermediate attachment for detachably connecting to the main body, the intermediate attachment comprising an inlet for receiving the airflow from the main body, and an outlet for emitting the airflow; and a hair treatment attachment for detachably connecting to the intermediate attachment, the hair treatment attachment configured to receive the airflow from the outlet of the intermediate attachment, and emit the airflow towards hair to be treated in use, wherein the intermediate attachment comprises a sensor module for sensing a property of at least one of hair to be treated and the hair treatment attachment.

This may enable sensing functionality (for example sensing a temperature of hair to be treated) to be provided to the haircare appliance without either the main body or the hair treatment attachment comprising a sensor module. The intermediate attachment may be retrofitted to an existing haircare appliance that previously did not have sensing functionality, for example with the intermediate attachment located between the main body and the hair treatment attachment. Additionally, as it may be desirable to use multiple different hair treatment attachments to achieve different hair treatments, e.g. to dry hair or curl hair, sensing functionality may be provided for multiple hair treatment attachments without the need to install sensing modules in each of the hair treatment attachments. This may reduce the cost of the hair treatment attachments and/or the haircare appliance. Furthermore, the intermediate attachment may remain connected to the main body whilst the hair treatment attachment is replaced. This may provide ease of use, for example by allowing a user to simply swap between hair treatment attachments without having to remove the intermediate attachment from the main body. Servicing of the haircare appliance may be made easier because a faulty sensor module may be replaced by replacing the intermediate attachment rather than, for example, disassembling the main body.

The hair treatment attachment may be configured to receive the airflow from the outlet of the intermediate attachment, and emit the airflow towards hair to be treated, such that a pre-determined hair treatment, for example a particular form of drying or styling, is achieved. The hair treatment attachment may comprise a treatment outlet through which the airflow is emitted towards hair to be treated, and the treatment outlet may be shaped to achieve the pre-determined hair treatment. The hair treatment attachment may comprise a plurality of treatment outlets. The hair treatment attachment may comprise any of a concentrator nozzle for increasing the velocity of the airflow, a diffuser nozzle for reducing the velocity of the airflow, a coanda surface nozzle for attracting hair to a surface of the coanda surface nozzle (for example to produce curls or hide fly away hair), or a comb nozzle for combing hair as it is dried.

The haircare appliance may comprise a controller for automatically controlling the airflow generator based on an output of the sensor module. Thereby, an amount of user input required to control the haircare appliance may be reduced compared to, for example, a haircare appliance where the user manually controls the airflow generator. Additionally, by controlling the airflow generator based on the output of the sensor module, the performance of the haircare appliance may be improved. For example, the controller may turn the airflow generator off when the output of the sensor indicates that the hair has been treated, which may reduce damage to the hair and reduce power usage. The controller may control the power or the speed of the airflow generator, which may adjust the airflow rate of the airflow.

The controller may be located in the intermediate attachment, and the intermediate attachment may be configured to communicate control data from the controller to the airflow generator. As a result, automatic control of the airflow generator based on an output of the sensor module may be added to an existing haircare appliance that did not previously have this functionality.

The controller may be located in the main body, and the intermediate attachment may be configured to communicate the output of the sensor module to the controller. As an existing haircare appliance comprising a hair treatment attachment and a main body may already comprise a controller, this may reduce the cost of the intermediate attachment as duplication of controllers may not be required to achieve the sensor-based control when retrofitting the intermediate attachment to the existing haircare appliance. The intermediate attachment may be configured to wirelessly transmit the output of the sensor module to the controller. This may remove the need for a physical electrical connection between the intermediate attachment and the main body for communicating the output. This may allow for a simpler connection between the intermediate attachment and the main body. The intermediate attachment may comprise a Bluetooth® module for communicating the output of the sensor module to the controller.

The haircare appliance may comprise a heater for heating the airflow and a heater controller for controlling the heater based on the output of the sensor module. Providing a heater may increase the range of treatment options deliverable by the haircare appliance and may enhance those treatment options, e.g. by providing quicker hair drying times. The heater controller may be configured to automatically control the heater. This may reduce an amount of user input required to control the haircare appliance compared to, for example, a haircare appliance where user input is required in response to the output of the sensor module.

The intermediate attachment may comprise a wireless transmitter for transmitting data representative of the output of the sensor module to a remote user device. This may enable sensor data to be transmitted to, and in some examples processed by, the remote user device. In some examples, the output may be displayed to a user via the remote user device. The remote user device may comprise any of a smart phone, a tablet, a smart watch and a further haircare appliance. The wireless transmitter may comprise a Wi-Fi™ transmitter or a Bluetooth® transmitter.

The intermediate attachment may comprise a wireless receiver for receiving command data, for example from the remote user device, and the airflow generator may be controllable based on the command data. Thereby the operation of the airflow generator may be controlled by the remote user device. The controller may control the airflow generator based on the command data. The heater controller may control the heater based on the command data. Thereby the operation of the heater may be controlled by the remote user device. The wireless receiver may comprise a Wi-Fi™ receiver or a Bluetooth® receiver.

The sensor module may comprise an optical sensor. As an optical sensor may provide a contactless sensing capability, the sensor module may be spaced from hair to be treated. This may facilitate the sensor module being located in the intermediate attachment rather than the hair treatment attachment.

The sensor module may comprise a distance sensor for sensing a distance between hair to be treated and the haircare appliance. Thereby, the function of the haircare appliance may be controlled based on a distance to the hair to be treated. For example, the haircare appliance may only operate the airflow generator when hair to be treated is within a threshold distance to ensure the airflow generator only operates when effective hair treatment may be achieved. This may reduce power consumption.

The sensor module may comprise a moisture sensor for sensing a moisture content of hair to be treated. As a result, over drying of hair to be treated may be inhibited. This may reduce a risk of damage to hair.

The sensor module may comprise a temperature sensor for sensing a temperature of hair to be treated. This may enable the haircare appliance to operate such that the hair is maintained within an appropriate treatment temperature range. Thereby, a risk of damage to hair by overheating or excessive treatment times due to insufficient temperature may be reduced.

The sensor module may comprise a sensor for sensing a type of hair treatment attachment connected to the intermediate attachment. Thereby the operation of the haircare appliance, for example the speed of the airflow generator, may be adapted to suit the type of hair treatment attachment. This may result in reduced power consumption and May reduce acoustic emissions. The sensor module may comprise an RFID reader and the hair treatment attachment may comprise a tag for reading by the RFID reader.

The sensor module may comprise at least one of an IR sensor, a radar sensor or an ultrasound sensor. These may have the benefit that any sensing emissions, for example emitted radar waves, emitted by the sensors may not be perceived by a user of the device. Additionally or alternatively, a radar sensor may have the benefit that it may not require a clear line of sight to hair to be treated. This may enable the radar sensor to sense the hair without the hair treatment attachment being configured to enable a clear line of sight between the radar sensor and the hair, which may simplify the design of the hair treatment attachment.

The sensor module may comprise an imaging sensor for capturing an image of hair to be treated. Thereby the sensor module may be able to detect the presence of hair within the image, and the haircare appliance may only operate the airflow generator or heater when hair is detected in the image. This may reduce the power usage of the haircare appliance. Additionally or alternatively, this may enable a property of hair to be treated, such as the colour or type of the hair, to be determined. As a result, the operation of the haircare appliance may be adapted based on the detected hair property, which may result in improvements to the effectiveness of the hair treatments for different types of hair.

The haircare appliance may comprise a user feedback module configured to provide feedback to a user based on an output of the sensor module. By providing feedback to the user, the user may be provided with a greater understanding of the state of the hair to be treated, and therefore may be more able to effectively treat the hair. For example, the user feedback module may output a signal to alert the user when a temperature of the hair exceeds a threshold. In response, the user may stop using the haircare appliance, or adjust the heater, to prevent damage to the hair.

The user feedback module may be located in the intermediate attachment. As a result, the functionality provided by the feedback module may be added to an existing haircare appliance without modification of the main body or hair treatment attachment.

The intermediate attachment may comprise a feedback controller for controlling the user feedback module based on the output of the sensor module. The user feedback module may be located in the main body, and the feedback controller may be configured to communicate control instructions to the user feedback module. Thereby the benefits provided by having a feedback module configured to provide feedback to a user based on an output of the sensor module may be achieved at a reduced cost if the intermediate attachment is attached to a main body that already comprises a user feedback module. In particular, the cost of the intermediate attachment may be reduced as duplication of the user feedback module may be avoided. For example, the main body may comprise a display, and the feedback controller may control the display to provide feedback to the user.

The user feedback module may comprise at least one of a haptic feedback module, a visual feedback module, and an aural feedback module. The haptic feedback module and the aural feedback module may be beneficial in that feedback may be provided to the user irrespective of if the user can see the haircare appliance. A visual feedback module, for example a display, may be beneficial as granular feedback, such as a numerical value for a temperature of hair to be treated, may be provided to the user.

The main body may comprise a first inductive coil, and the intermediate attachment may comprise a second inductive coil for inductively coupling to the first inductive coil. This may enable wireless energy and data transfer between the main body and the intermediate attachment. This may have two benefits. Firstly, this may provide a simpler connection, compared to a physical electrical connection, between the intermediate attachment and the main body. Secondly, this may enable the cost and weight of the intermediate attachment to be reduced as the intermediate attachment may be able to use power provided by the main body rather than requiring a dedicated power source within the intermediate attachment.

The intermediate attachment may comprise a power source configured to power the sensor module. As a result, the intermediate attachment may be attached to a main body which does not comprise a mechanism for transferring power to the intermediate attachment.

The intermediate attachment may comprise a locking feature for inhibiting rotation of the hair treatment attachment relative to the intermediate attachment. As a result, a correct orientation of the sensor module relative to the hair treatment attachment may be ensured, which may prevent a view of the hair by the sensor module from being obscured by the hair treatment attachment.

The intermediate attachment may comprise a flow deflector (for example, a moveable baffle) for deflecting the airflow in use. As a result, the capability to deflect the airflow, and thereby control the airflow delivered to the user, may be added to an existing haircare appliance that did not previously have this functionality. The intermediate attachment may comprise a flow deflector controller which may control the flow deflector based on the output of the sensor module. For example, if the sensor module output indicates that a first type of hair treatment attachment is connected to the intermediate attachment, then the flow deflector controller may move the flow deflector to a first position in which the airflow is not diverted. If the sensor module output indicates that a second type of hair treatment attachment is connected to the intermediate attachment, then the flow deflector controller may move the flow deflector to a second position in which the airflow is diverted. Thereby, control of the airflow based on the output of the sensor may be achieved without communication with the main body.

The intermediate attachment may comprise a throttle (for example a valve) for throttling the airflow in use. As a result, the capability to throttle the airflow, and thereby control the airflow delivered to the user, may be added to an existing haircare appliance that did not previously have this functionality. The intermediate attachment may comprise a throttle controller which may control the throttle based on the output of the sensor module. As a result, throttling of the airflow based on the output of the sensor may be achieved without communication with the main body.

The sensor module may be located outside of the airflow flowing through the intermediate attachment in use. Thereby disturbance of the airflow by the sensor module may be prevented. The operational life of the sensor module may also be prolonged as the sensor module may not be exposed to the potentially heated airflow which may have otherwise degraded the sensor module. The intermediate attachment may comprise a first portion for defining an airflow path between the inlet and the outlet, a second portion that extends away from the first portion, and the sensor module may be located in the second portion. This may ensure the sensor module is located outside of the airflow and may provide a convenient arrangement as the second portion may perform a secondary function as the locking feature.

The intermediate attachment may comprise a first connection portion for detachably connecting the intermediate attachment to the main body, and a second connection portion for detachably connecting the intermediate attachment to the hair treatment attachment. This may provide a simpler arrangement that may be less prone to failure than, for example, an arrangement with separate connecting structures detachably attached to the intermediate attachment. At least one of the first or second sections may comprise one of a magnetic connection mechanism and a snap fit connection mechanism. This may provide a simple mechanism for connecting the intermediate attachment to the main body or the hair treatment attachment to the intermediate attachment that may allow the user to remove and connect the intermediate attachment quickly and easily.

The first connection portion may match a connection portion of the hair treatment attachment and the second connection portion may match a connection portion of the main body. As a result, the intermediate attachment may attach between the main body and the hair treatment attachment, and the intermediate attachment may be disconnected, and the main body connected directly to the hair treatment attachment. For example, the first connection portion may comprise projections that match projections of the connection portion of the hair treatment attachment, and the second connection portion may comprise slots for receiving the projections that match slots of the connection portion of the main body.

The inlet may be opposite the outlet, and the intermediate attachment may be configured such that the airflow follows a linear path from the inlet to the outlet in use. This may result in a higher flowrate being achieved for the airflow due to reduced pressure losses when compared to, for example, an intermediate attachment where the inlet is perpendicular to the outlet, or the airflow follows a contorted flow path.

The intermediate attachment may have a length measured between the inlet and the outlet, and the length may be less than at least one of a maximum dimension of the main body in a direction parallel to the length when the main body is connected to the intermediate attachment and a maximum dimension of the hair treatment attachment in a direction parallel to the length when the hair treatment attachment is connected to the intermediate attachment. A more compact intermediate attachment may therefore be achieved. This may reduce the impact the intermediate attachment may have on the usability of an existing haircare appliance, which may have been designed to have a specific, easy to use, length.

The intermediate attachment may comprise an annular shape. This may provide a compact arrangement for the intermediate attachment as the airflow may flow through a hollow centre of the annular shape, and the sensor module may be arranged around an outside portion of the annular shape.

The haircare appliance may comprise a further hair treatment attachment different to the hair treatment attachment, and the hair treatment attachment and further hair treatment attachment may be interchangeably connectable to the intermediate attachment. This may allow for different functionality to be provided by the hair treatment attachments whilst still realising the benefits provided by the intermediate attachment.

The haircare appliance may comprise a circuitry housing remote from the main body. The controller may be located in the circuitry housing, and a wire may extend from the circuitry housing to the main body to couple the airflow generator to the controller. An additional wire extending from the circuitry housing to the main body may couple the controller to the heater.

The present invention also provides an intermediate attachment for a haircare appliance, the intermediate attachment comprising a first connection portion for detachably connecting to a main body of the haircare appliance; a second connection portion for detachably connecting to a hair treatment attachment; an inlet for receiving an airflow from the main body; an outlet for emitting the airflow into the hair treatment attachment such that the airflow is emitted from the hair treatment attachment towards hair to be treated in use; and a sensor module for sensing a property of at least one of hair to be treated and the hair treatment attachment.

As discussed above, this then provides an intermediate attachment that may be retrofitted to an existing haircare appliance that previously did not have sensing functionality.

Thereby the functionality of the existing haircare appliance may be enhanced without modification if the existing haircare appliance had already been manufactured or redesigning if the existing haircare appliance had already been designed but had yet to be manufactured.

### BRIEF DESCRIPTION OF THE DRAWINGS

FIG. 1 is a perspective view of a haircare appliance according to the present invention;

FIG. 2 is an exploded view of the haircare appliance of FIG. 1;

FIG. 3 is a cross-sectional view of a main body of the haircare appliance of FIG. 1;

FIG. 4 is a block diagram illustrating communication between electrical components of the haircare appliance of FIG. 1;

FIG. 5 is a perspective view of an intermediate attachment of the haircare appliance of FIG. 1;

FIG. 6 is a perspective view of a hair treatment attachment of the haircare appliance of FIG. 1;

FIG. 7 is a front view of a second embodiment of a haircare appliance according to the present invention;

FIG. 8 is a partially exploded perspective view of the haircare appliance of FIG. 7;

FIG. 9 is a perspective view of a third embodiment of a haircare appliance according to the present invention;

FIG. 10 is an exploded view of the haircare appliance of FIG. 9;

FIG. 11 is a cross-sectional view of a main body of the haircare appliance of FIG. 9;

FIG. 12 is a perspective view of a fourth embodiment of a haircare appliance according to the present invention; and

FIG. 13 is an exploded view of the haircare appliance of FIG. 12.

### DETAILED DESCRIPTION OF THE INVENTION

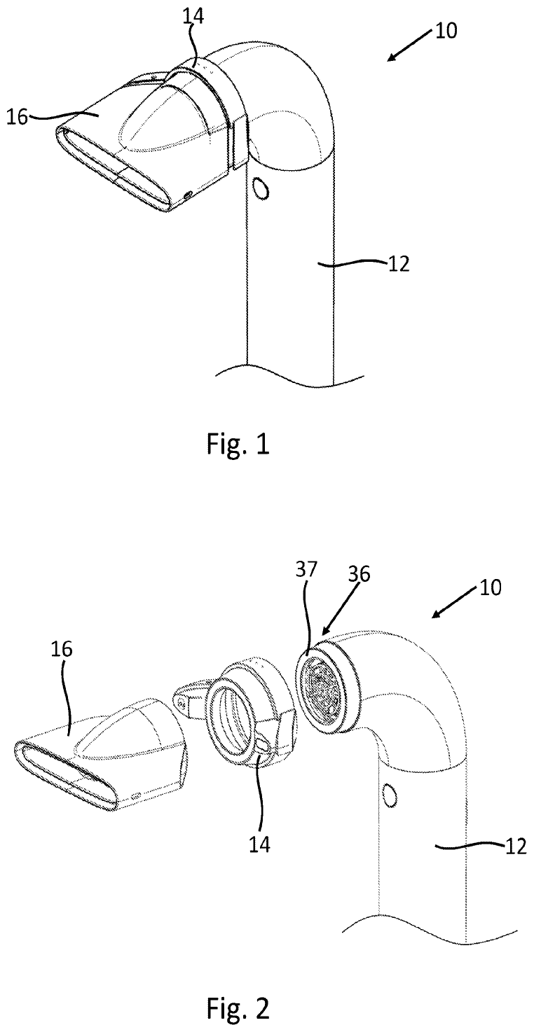

The haircare appliance 10 of FIGS. 1 and 2 comprises a main body 12, an intermediate attachment 14 and a hair treatment attachment 16. The intermediate attachment 14 is detachably connected between the main body 12 and the hair treatment attachment 16.

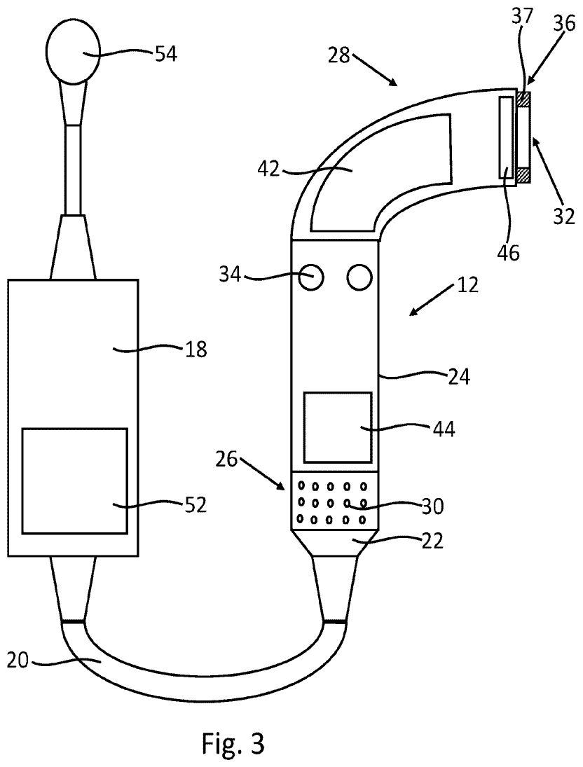

Turning now to FIG. 3, in this example, the main body 12 is connected to a circuitry housing 18 by an electrical cable 20 that extends from the circuitry housing 18 to the main body 12.

The main body 12 defines a hollow, generally elongate, handle that is intended to be grasped by a user in use. The main body 12 comprises a conical end portion 22 and a wall 24 extending upwardly from the conical end portion 22, such that a first end 26 of the main body 12 is generally cylindrical in form. The main body 12 has a second end 28 distal from the first end 26, and the main body 12 is curved such that the second end 28 is angled relative to the first end 26. An inlet 30 is located at the first end 26 of the main body 12 on the wall 24, and takes the form of a plurality of apertures, for example in a mesh-like structure. An outlet 32 is located at the second end 28, and comprises an aperture through which air may flow in use. The electrical cable 20 enters the main body 12 through an aperture formed in the conical end portion 22. A user interface 34 is formed on the wall 24 and may take the form of a plurality of buttons, a touchscreen, or a combination thereof.

A connection portion 36 surrounds the outlet 32 of the main body 12 and detachably connects the main body 12 to the intermediate attachment 14. The connection portion 36 comprises a magnet 37 for detachably connecting to a magnet of a first connection portion 38 of the intermediate attachment 14 or a magnet of a connection portion 40 of the hair treatment attachment 16. Thereby, the main body 12 may be detachably connected to either the intermediate attachment 14 or the hair treatment attachment 16. An external diameter of the connection portion 36 is sized such that the connection portion 36 may be inserted into the inlet 60 of the intermediate attachment 14 or the inlet 82 of the hair treatment attachment 16, which may improve the robustness of the connection between the main body 12 and the intermediate attachment 14 or the hair treatment attachment 16.

Disposed within the main body 12 is a heater 42, an airflow generator 44 and a first inductive coil 46. The airflow generator 44 comprises an impeller driven by an electric motor. The airflow generator 44 generates an airflow between inlet 30 and the outlet 32 of the main body 12. The heater 42 comprises heating elements to optionally heat the airflow.

The first inductive coil 46 is located towards the second end 28 of the main body 12 and inductively couples with a second inductive coil 48 that is located in the intermediate attachment 14, such that power and data may be transferred between the main body 12 and the intermediate attachment 14. As will be described in more detail below, the intermediate attachment 14 comprises a sensor module 50 for sensing a property of hair to be treated (for example, temperature, moisture, type or presence of the hair), and the second inductive coil 48 transmits data comprising an output of the sensor module 50 (for example a value representing the sensed property of the hair) to the first inductive coil 46. The first inductive coil 46 is connected to a controller 52 located in the circuitry housing 18 via a wire in the electrical cable 20. The first inductive coil 46 communicates data to the controller 52 such that the output of the sensor module 50 is communicated to the controller 52. In other embodiments, the first 46 and second 48 inductive coil may be replaced by another wireless transmitter and receiver pair such as a Bluetooth® or Wi-Fi™ transmitter and receiver. Equally, embodiments are envisaged in which a wired connection communicates the data between the intermediate attachment 14 and the main body 12. For example, the wired connection may comprise a pair of electrical contacts, one on each of the intermediate attachment 14 and the main body 12, configured to enable the wired connection to be detachably connectable.

The circuitry housing 18 defines an enclosure that houses the controller 52. A power connector 54 in the form of a plug is coupled to the opposite side of the circuitry housing 18 to the electrical cable 20. The power connector 54 is configured to interact with an AC mains power supply, for example via a mains socket, to provide electrical current to the haircare appliance 10 in use.

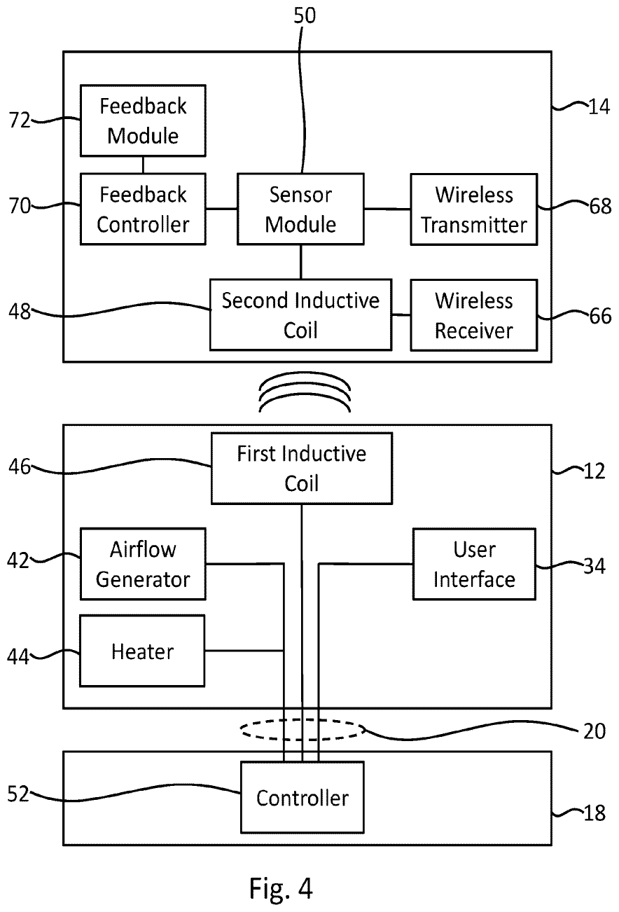

As shown in FIG. 4, the controller 52 is connected to the first inductive coil 46, the user interface 34, the airflow generator 42 and the heater 44 by wires within the electrical cable 20. The controller 52 is responsible for controlling the airflow generator 42 and the heater 44 in response to inputs from the user interface 34. For example, in response to inputs from the user interface 34, the controller 52 may control the power or the speed of the airflow generator 42 in order to adjust the airflow rate of the airflow, and the power of the heater 44 in order to adjust the temperature of the airflow.

The controller 52 also automatically controls the airflow generator 42, and in some examples the heater 44, based on data communicated by the inductive coils 46,48 and thereby the output of the sensor module 50. As described previously, the sensor module 50 senses a property of the hair to be treated. Therefore, the controller 52 automatically controls the airflow generator 42, and in some examples the heater 44, based on a property of the hair. In one example, the sensor module 50 may comprise a moisture sensor for sensing a moisture content (the property) of hair to be treated, and the controller 52 may operate the airflow generator 42 at a first speed when the moisture level is above a moisture threshold representing wet hair, and the controller 52 may operate the airflow generator 42 at a second, lower speed when the moisture level is below the moisture threshold. Thereby, over drying of hair to be treated may be inhibited. In another example, the sensor module 50 may comprise a temperature sensor for sensing a temperature (the property) of hair to be treated, and the controller 52 may operate the heater 44 at a first level when the temperature of the hair is below a temperature threshold, and then operate the heater at a second, lower level when the temperature of the hair is above the temperature threshold. As a result, a risk of damage to the hair by overheating may be reduced. In a further example, the sensor module 50 may comprise a distance sensor for sensing a distance between hair to be treated and the haircare appliance 10. In this embodiment, the sensor module 50 comprises an optical sensor, for example an IR sensor, which provides a contactless sensing capability. An IR sensor may be used to sense a temperature of the hair. Other embodiments are envisaged in which the sensor module 50 comprises a radar sensor or an ultrasound sensor, which may be used to sense a distance between the haircare appliance 10 and the hair. The sensor module 50 may comprise a plurality of sensors of different types. It is also envisaged that the heater 44 may be controlled by a separate heater controller located in the main body 12 that controls the heater 44 based on the output of the sensor module 50.

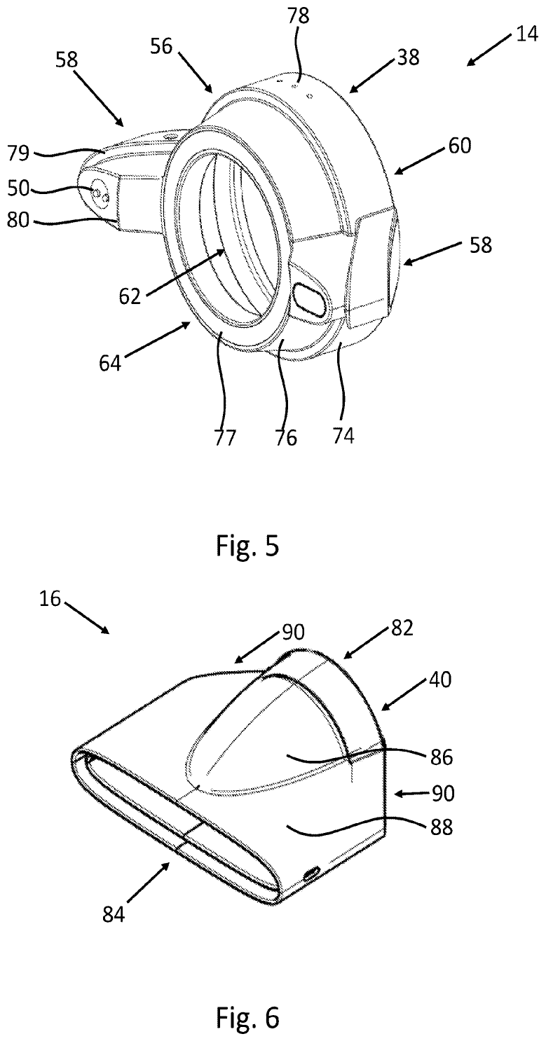

Turning now to FIGS. 4 and 5, the intermediate attachment 14 comprises a main body 56 and a pair of projecting portions 58.

The main body 56 of the intermediate attachment 14 comprises an inlet 60, an outlet 62, the first connection portion 38, a second connection portion 64, the second inductive coil 48, a wireless receiver 66, a wireless transmitter 68, a feedback controller 70 and a feedback module 72.

The main body 56 of the intermediate attachment 14 has an annular shape with the inlet 60 of the intermediate attachment 14 at one end for receiving the airflow from the main body 12 and the outlet 62 of the intermediate attachment 14 at the other, opposite, end for emitting the airflow into the hair treatment attachment 16. The main body 56 of the intermediate attachment 14 is shaped such that in use the airflow follows a linear path between the inlet 60 and the outlet 62 of the intermediate attachment 14. The main body 56 of the intermediate attachment 14 comprises a first portion 74, located towards the inlet 60 of the intermediate attachment 14, with a first external diameter and a second portion 76, located towards the outlet 62 of the intermediate attachment 14, with a second, smaller external diameter. Thereby, the second portion 76 may be inserted into the inlet 82 of the hair treatment attachment 16, which may improve the robustness of the connection between the hair treatment attachment 16 and the intermediate attachment 14.

The first connection portion 38 is located towards the inlet 60 of the intermediate attachment 14 and detachably connects the intermediate attachment 14 to the main body 12. The first connection portion 38 comprises a magnet for detachably connecting to the magnet 37 of the connection portion 36 of the main body.

The second connection portion 64 surrounds the outlet 62 of the intermediate attachment and is located on an outer face of the second portion 76. The second connection portion 64 comprises a magnet 77, shown here as an annular magnet, for detachably connecting to the magnet of the connection portion 40 of the hair treatment attachment 16.

The second inductive coil 48 is located towards the inlet 60 of the main body 56 of the intermediate attachment 14 and is connected to the sensor module 50 and the wireless receiver 66 such that the data transmitted to the first inductive coil 46 by the second inductive coil 48 comprises outputs from both the sensor module 50 and the wireless receiver 66.

The wireless transmitter 68 is connected to the sensor module 50 and transmits data comprising the output of the sensor module 50 to a remote user device. In one example, the remote user device may comprise a phone running a suitable application. The output of the sensor module 50 may then be displayed to a user via the phone, and the user may then alter the operation of the haircare appliance 10 based on the displayed output. For example, the temperature of the hair to be treated may be displayed, and the user may alter the speed of the airflow generator 42 using the user interface 34 based on the displayed temperature. In other examples, the remote user device may comprise a tablet, a smart watch or a further haircare appliance. In some examples, the wireless transmitter 68 may also perform the function of the second inductive coil 48, and the main body may comprise a wireless receiver instead of the first inductive coil 46 for receiving the data communicated by the wireless transmitter 68. In some examples, the wireless transmitter may comprise a Wi-Fi™ transmitter or a Bluetooth® transmitter.

The wireless receiver 66 is connected to the second inductive coil 48. The wireless receiver 66 receives command data from a remote user device, which may be the same or a different remote user device to that which communicates with the wireless transmitter 68. The command data is communicated to the controller 52 via the inductive coils 46,48. The controller 52 controls the airflow generator 42, and in some examples the heater 44, based on the command data. In some examples, the wireless receiver may comprise a Wi-Fi™ receiver or a Bluetooth® receiver.

The feedback controller 70 is connected to the sensor module 50 and the feedback module 72 and controls the feedback module 72 based on the output of the sensor module 50. The feedback module 72 provides feedback to a user. Thereby, feedback is provided to the user based on a property of the hair to be treated. In this embodiment, the feedback module 72 comprises a plurality of LEDs 78 located on the outside of the main body 56 of the intermediate attachment 14. The LEDs 78 provide visual feedback to the user. For example, the feedback controller 70 may turn the LEDs 78 on when a temperature of the hair exceeds the temperature threshold. In response, the user may stop using the haircare appliance 10 or adjust the heater 44 using the user interface 34 to inhibit damage to the hair. In other examples, the feedback module 72 may comprise one of a haptic feedback module (for example, a motor that vibrates to provide feedback) or an aural feedback module (for example, a speaker which emits an audible tone to provide feedback). Although in this embodiment the feedback module 72 is located in the intermediate attachment 14, it is conceivable that the feedback module 72 may be located in the main body 12. The feedback controller 70 may then send control instructions to the feedback module 72 via a wireless or wired communications device, such as the inductive coils 46,48.

Each projecting portion 58 comprises a body section 79 and a locking section 80. Each body section 79 extends away from an opposite side of the main body 56 of the intermediate attachment 14 and is angled towards the hair treatment attachment 16. Each locking section 80 extends towards the hair treatment attachment 16 from a side of each body section 79 that faces the hair treatment attachment 16. The sensor module 50 is located in a face of one of the locking sections 80 that faces the hair treatment attachment 16. Thereby, the sensor module 50 is located outside of the airflow flowing through the intermediate attachment 14 in use.

The locking sections 80 have a lesser height than the body sections 79 and are sized such that the locking portions 80 may be inserted into corresponding sections of the hair treatment attachment 16 and thereby inhibit rotation of the hair treatment attachment 16 relative the intermediate attachment 14. As a result, a correct orientation of the sensor module 50 relative to the hair treatment attachment 16 may be ensured, which may inhibit a view of the hair by the sensor module 50 from being obscured by the hair treatment attachment 16.

Although in this embodiment the intermediate attachment 14 comprises a single sensor module 50, it is conceivable that the intermediate attachment 14 may comprise a plurality of sensor modules. For example, a sensor module 50 located in each projecting portion 80.

The sensor module 50 takes the form previously described and is connected to the feedback controller 70, the wireless transmitter 68, and the second inductive coil 48. The sensor module 50 senses a property of hair to be treated and provides the output to the second inductive coil 48, feedback controller 70, and wireless transmitter 68.

The intermediate attachment 14 has a length measured between the inlet 60 and the outlet 62 of the intermediate attachment 14. The length is less than a maximum dimension of the main body 12 in a direction parallel to the length when the main body 12 is connected to the intermediate attachment 14. In particular, the maximum dimension of the main body 12 is measured between the outlet 32 of the main body and a surface of the wall 24 on the opposite side of the main body 12 to the outlet 32 of the main body 12. The length is also less than a maximum dimension of the hair treatment attachment 16 in a direction parallel to the length when the hair treatment attachment 16 is connected to the intermediate attachment 14. In particular, the maximum dimension of the hair treatment attachment 16 is measured between an inlet 82 and the outlet 84 of the hair treatment attachment 16.

Turning now to FIG. 6, the hair treatment attachment 16 comprises the inlet 82, the outlet 84 and the connection portion 40. The hair treatment attachment 16 has an inlet section 86 which connects to an outlet section 88. The inlet 82 of the hair treatment attachment 16 is located in an end of the inlet section 86 and has a circular shape. The inlet 82 of the hair treatment attachment 16 receives the airflow from the intermediate attachment 14. The outlet 84 is oval shaped and is located in an end of the outlet section 88. The outlet 84 emits the airflow towards hair to be treated such that a pre-determined hair treatment, in this embodiment drying, is achieved. In this embodiment, the outlet 84 of the hair treatment attachment 16 has a smaller cross-sectional area than the inlet 82 of the hair treatment attachment 16 such that the hair treatment attachment 16 may be regarded as comprising a concentrator nozzle for increasing the velocity of the airflow. The connection portion 40 of the hair treatment attachment 16 is located towards the inlet 82 of the hair treatment attachment 16 and detachably connects the hair treatment attachment to the intermediate attachment 14 or the main body 12. As discussed previously, the connection portion 40 of the hair treatment attachment 16 comprises a magnet for detachably connecting to the magnet 77 of the second connection portion 64 of the intermediate attachment 14 or the magnet 37 of the connection portion 36 of the main body. A side of the outlet section 88 which faces the intermediate attachment 14 comprises two openings 90 sized to receive the locking sections 80 of the intermediate attachment 14. In addition to providing the locking functionality discussed above, this enables a clear line of sight for the sensor module 50, through the hair treatment attachment 16, to the hair to be treated.

To assemble the haircare appliance 10, the user connects the magnet 37 of the connection portion 36 of the main body 12 to the magnet of the first connection portion 38 of the intermediate attachment 14 to connect the main body 12 to the intermediate attachment 14. The user then aligns the locking sections 80 of the intermediate attachment 14 with the openings 90 of the hair treatment attachment 16 and connects the magnet 77 of the second connection portion 64 of the intermediate attachment 14 to the magnet of the connection portion 40 of the hair treatment attachment 16 to attach the intermediate attachment 14 to the hair treatment attachment 16.

To attach a different hair treatment attachment to the haircare appliance 10 the user disconnects the hair treatment attachment 16 from the intermediate attachment 14 and connects a different hair treatment attachment to the second connection portion 64 of the intermediate attachment 14. Attachment of a different hair treatment attachment may be desirable as different hair treatment attachments may provide different functionality, for example enabling a different pre-determined hair treatment, such as hair curling, to be achieved.

By providing the haircare appliance 10 with the intermediate attachment 14, sensing functionality is provided to the haircare appliance without either the main body 12 or the hair treatment attachment 16 comprising a sensor module 50. The intermediate attachment 14 may be retrofitted to an existing haircare appliance that previously did not have sensing functionality. Additionally, as it may be desirable to use multiple different hair treatment attachments to achieve different hair treatments, sensing functionality may be provided for multiple hair treatment attachments without the need to install sensing modules in each of the hair treatment attachments. This may reduce the cost of the hair treatment attachments and/or the haircare appliance. Furthermore, the intermediate attachment 14 may remain connected to the main body 12 whilst the hair treatment attachment 16 is replaced. This may provide ease of use, for example by allowing a user to simply swap between hair treatment attachments without having to remove the intermediate attachment 14 from the main body. Servicing of the haircare appliance 10 may be made easier because a faulty sensor module 50 may be replaced by replacing the intermediate attachment 14 rather than, for example, disassembling the main body 12.

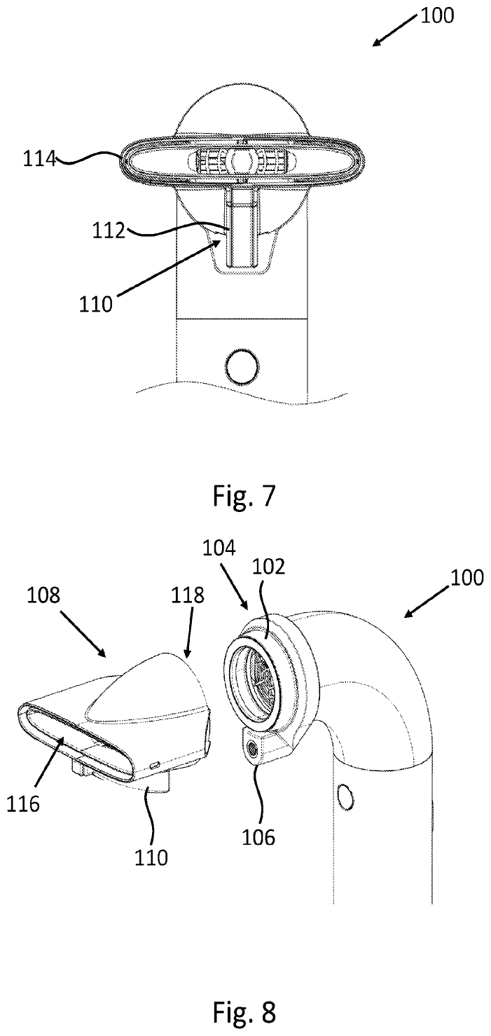

FIGS. 7 and 8 show a further example of a haircare appliance 100. The haircare appliance 100 is similar to that described above but with three exceptions. Firstly, the main body 102 of the intermediate attachment 104 comprises a single projecting portion 106 that extends in a perpendicular direction from a lower surface of the main body 102 of the intermediate attachment 104. The sensor module is located within a face of the projecting portion 106 that faces the hair treatment attachment 108. Secondly, the projecting portion 106 does not have a distinct locking section. Instead, the projecting portion 106 performs the locking function as will be described subsequently. Thirdly, the hair treatment attachment 108 comprises a locking body 110. The locking body 110 comprises a pair of parallel walls 112 that project in a perpendicular direction from a centre of a lower surface of the outlet section 114. The walls 112 extend around an underside of the hair treatment attachment 108, from the outlet 116 to adjacent to the inlet 118. The locking body 110 is sized such that the projecting portion 106 can be received between the walls 112 of the locking body 110 to inhibit rotation of the hair treatment attachment 108 relative the intermediate attachment 104.

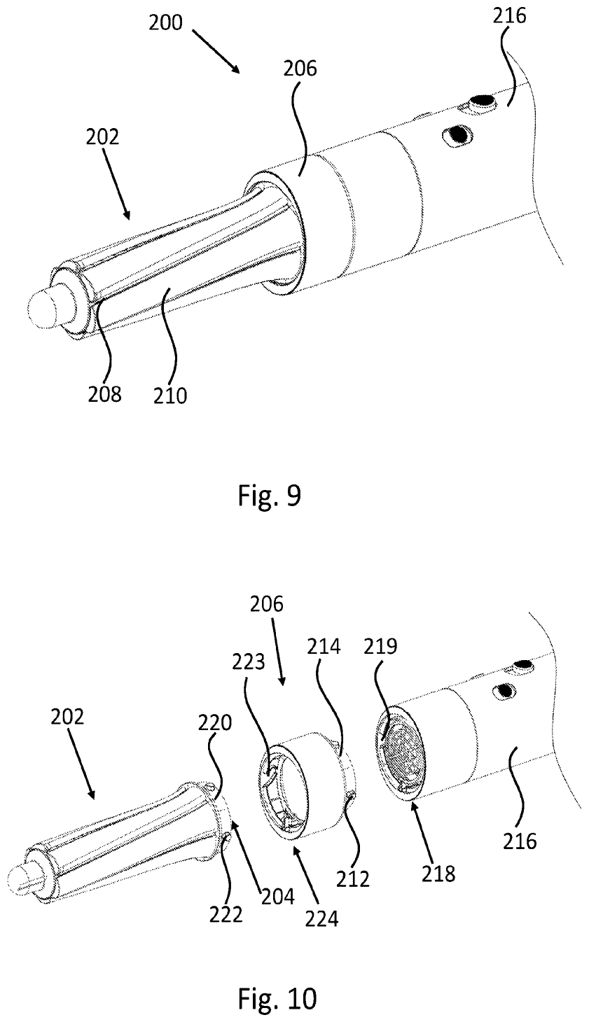

Although the hair treatment attachment 16 is described above as comprising a concentrator nozzle, other forms of hair treatment attachment are envisaged. For example, the hair treatment attachment may comprise a diffuser nozzle for reducing the velocity of the airflow, a coanda surface nozzle for attracting hair to a surface of the coanda surface nozzle (for example to produce curls or hide fly away hair), or a comb nozzle for combing hair as it is dried. FIGS. 9, 10 and 11 show an alternative example of a hair care appliance that comprises a coanda surface nozzle.

The haircare appliance 200 of FIGS. 9, 10 and 11 is similar to that described above with reference to FIGS. 1 to 6 but with the following exceptions. Firstly, the hair treatment attachment 202 of FIGS. 9 and 10 is generally cylindrical in form, is open at one end, and closed at the other end. The open end serves as an inlet 204 into the hair treatment attachment 202 for receiving the airflow from the intermediate attachment 206. The hair treatment attachment 202 also comprises a plurality of hair treatment outlets 208. The hair treatment outlets 208 have a slot shape and extend in a longitudinal direction. The hair treatment outlets 208 are circumferentially spaced from one another and are arranged around the full circumference of the hair treatment attachment 202. The hair treatment outlets 208 are angled with respect to an outside surface 210 of the hair treatment attachment 202 such that the airflow is emitted from the hair treatment outlets 208 in a circumferential direction. Thereby, hair brought into the vicinity of the surface 210 of the hair treatment attachment 202 is entrained within the airflow and wrapped around the hair treatment attachment 202 due to the coanda effect. This may provide a curling treatment to the hair.

Secondly, an alternative connection approach is employed to the magnets employed in the examples of FIGS. 1 to 8. The intermediate attachment 206 of FIGS. 9 and 10 comprises connection projections 212 that project perpendicular to, and from, the outside of the first connection portion 214 of the intermediate attachment 206. The connection projections 212 are circumferentially spaced by 90° from each other around the first connection portion 214 of the intermediate attachment 206.

The main body 216 comprises connection slots 219 located in the inside of the connection portion 218 of the main body 216 that are circumferentially spaced from each other by 90°. The connection slots 219 of the main body 216 are sized to receive the connection projections 212 to connect the main body 216 to the intermediate attachment 206.

The connection portion 220 of the hair treatment attachment 202 comprises connection projections 222 that match the connection projections 212 of the intermediate attachment 206. The second connection portion 224 of the intermediate attachment 206 comprises connection slots 223 that match those of the main body 216. Thereby the main body 216 may be connected to the intermediate attachment 206 or the hair treatment attachment 202.

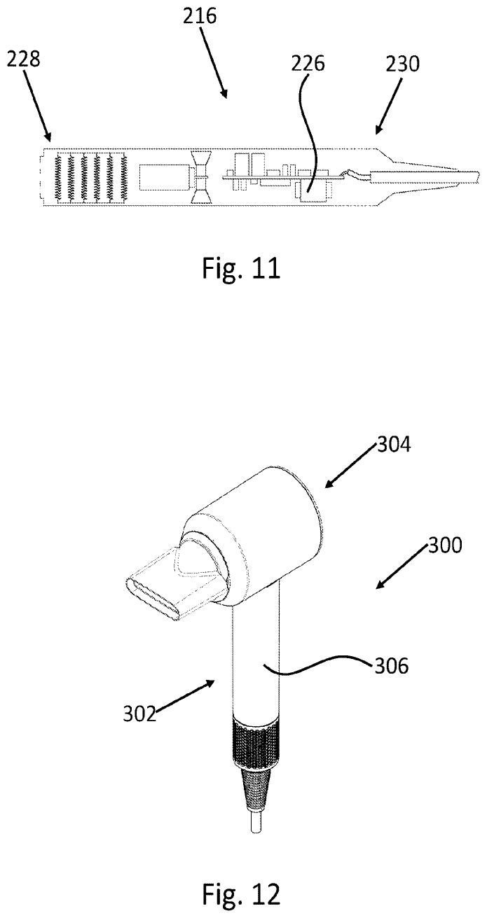

Thirdly, as shown in FIG. 11, the circuitry housing 18 and electrical cable 20 are omitted. Instead, the controller 226 is located within the main body 216. Additionally, the main body 216 is straight such that the second end 228 is not angled in relation to the first end 230.

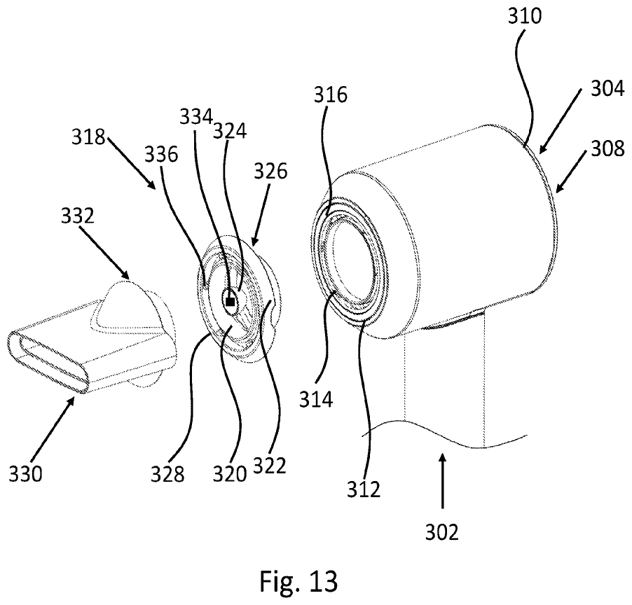

FIGS. 12 and 13 show an alternative example of a haircare appliance 300. The haircare appliance 300 is similar to that described above but with several exceptions.

The main body 302 is similar to the main body 216 described with reference to FIG. 11 except that the main body 302 comprises a head section 304 that forms the second end of the main body 302.

The head section 304 has a cylindrical shape and extends perpendicular to the main body 302. The head section 304 has a central bore 308 than extends between a rear face 310 and a front face 312 of the head section 304. The head section 304 has ducting for conveying the airflow around the bore 308 and to an aperture 314 of the head section 304. The aperture 314 of the head section emits the airflow from the head section 304 into the intermediate attachment 318 and therefore acts as the outlet of the main body 302. The aperture 314 of the head section comprises a circular slot in the front face 312 of the head section 304 that surrounds the bore 308. The airflow emitted from the aperture 314 of the head section 304 acts to induce an additional airflow through the bore 308. The head section 304 further comprises a magnet 316. The magnet 316 surrounds the aperture 314 of the head section 304 on the front face 312 of the head section 304. As will be discussed subsequently, the magnet 316 acts as the connection portion of the main body 302.

The intermediate attachment 318 comprises an inner portion 320, an outer portion 322, and a sensor housing 324. The inner portion 320 and the sensor housing 324 are located inside the bore 308 of the head section 304. The inner portion 320 has an outside diameter that is less than the diameter of the bore 308.

The outer portion 322 comprises a first magnet 326 located towards a face of the outer portion 322 that faces the head section 304 and a second magnet 328 located towards a face of the outer portion 322 that faces the hair treatment attachment 330. The outer portion 322 has a greater diameter than the inner portion 320 and surrounds, but is spaced from, the inner portion 320. The outer portion 322 has a length, measured perpendicular to the diameter, that is less than a length of the inner portion 320. The first magnet 326 extends around a circumference of the outer portion 322 and magnetically connects to the magnet 312 of the head section 304. Thereby the first magnet 326 may be considered the first connection portions of the intermediate attachment 318. The second magnet 328 extends around the circumference of the outer portion 322 and magnetically connects to a further magnet 332 located in the hair treatment attachment 330 (discussed subsequently). Thereby the second magnet 328 may be considered the second connection portions of the intermediate attachment 318.

The sensor housing 324 has a smaller diameter than the inner portion 320 and is surrounded by, but spaced from, the inner portion 320. The sensor module 334 is located in a face of the sensor housing 324 that faces the hair treatment attachment 330.

The inner portion 320, outer 322 portion, and sensor housing 324 are connected by a plurality of struts 336. There are gaps between the struts 336 such that the airflow may pass through the intermediate attachment 318.

As discussed above, the hair treatment attachment 330 comprises a further magnet 332 for magnetically connecting to the second magnet 328 of the intermediate attachment 318. The further magnet 332 is arranged circumferentially in a face of the hair treatment attachment 330 that faces the intermediate attachment 318.

In the embodiments discussed above, the sensor module senses a property of the hair to be treated. However, it is also envisaged that the sensor may sense a property of the hair treatment attachment. For example, the sensor may sense a type of hair treatment attachment connected to the intermediate attachment. In examples, the sensor module may comprise an RFID reader, and the hair treatment attachment may comprise a tag for reading by the RFID reader. The sensor module may then produce an output indicative of the type of hair treatment attachment connected to the intermediate attachment, and the controller may control the airflow generator based on the output. For example, the controller may operate the airflow generator at a first speed if a first type of hair treatment attachment is connected, and a second speed if a second type of hair treatment attachment is connected.

In the embodiments described above, the electrical components of the haircare appliance, for example the controller, are described as being in either the intermediate attachment, the main body, or the circuitry housing. However, it is conceivable that the electrical components may be distributed differently between the main body, the intermediate attachment, and the circuitry housing. For example, the controller may be located in the intermediate attachment and command data from the controller communicated to the airflow generator and heater via the indictive coils. Equally, the feedback module may be located in the main body, and the feedback controller located in the intermediate attachment may communicate commands to the feedback module via a wired or wireless device, such as the inductive coils.

Embodiments are also envisaged in which the intermediate attachment comprises a power source, for example a battery. In these embodiments, the intermediate attachment may also comprise a charging port for allowing recharging of the power source.

Embodiments are also envisaged in which the intermediate attachment comprises a flow deflector (for example, a moveable baffle) for deflecting the airflow in use. The intermediate attachment may comprise a flow deflector controller which may control the flow deflector based on the output of the sensor module. For example, if the sensor module output indicates that a first type of hair treatment attachment is connected to the intermediate attachment, then the controller may move the flow deflector to a first position in which the airflow is not diverted. If the sensor module output indicates that a second type of hair treatment attachment is connected to the intermediate attachment, then the controller may move the flow deflector to a second position in which the airflow is diverted. Thereby, control of the airflow based on the output of the sensor may be achieved without communication with the main body.

Embodiments are also envisaged in which the intermediate attachment comprises a throttle (for example a valve) for throttling the airflow in use. The intermediate attachment may comprise a throttle controller. The throttle controller may control the throttle based on the output of the sensor module. As a result, throttling of the airflow based on the output of the sensor may be achieved without communication with the main body.

1. A haircare appliance comprising:
a main body housing an airflow generator for generating an airflow;
an intermediate attachment for detachably connecting to the main body, the intermediate attachment comprising an inlet for receiving the airflow from the main body, and an outlet for emitting the airflow; and
a hair treatment attachment for detachably connecting to the intermediate attachment, the hair treatment attachment configured to receive the airflow from the outlet of the intermediate attachment, and emit the airflow towards hair to be treated in use,
wherein the intermediate attachment comprises a sensor module for sensing a property of at least one of the hair to be treated and the hair treatment attachment.

2. The haircare appliance as claimed in claim 1, wherein the haircare appliance comprises a controller for automatically controlling the airflow generator based on an output of the sensor module.

3. The haircare appliance as claimed in claim 2, wherein the controller is located in the intermediate attachment, and the intermediate attachment is configured to communicate control data from the controller to the airflow generator.

4. The haircare appliance as claimed in claim 2, wherein the controller is located in the main body, and the intermediate attachment is configured to communicate the output of the sensor module to the controller.

5. The haircare appliance as claimed in claim 1, wherein the haircare appliance comprises a heater for heating the airflow, and a heater controller for controlling the heater based on an output of the sensor module.

6. The haircare appliance as claimed in claim 1, wherein the intermediate attachment comprises a wireless transmitter for transmitting data representative of an output of the sensor module to a remote user device.

7. The haircare appliance as claimed in claim 1, wherein the intermediate attachment comprises a wireless receiver for receiving command data, and the airflow generator is controllable based on the command data.

8. The haircare appliance as claimed in claim 1, wherein the sensor module comprises at least one of:
a distance sensor for sensing a distance between the hair to be treated and the haircare appliance;
a moisture sensor for sensing a moisture content of the hair to be treated;
a temperature sensor for sensing a temperature of the hair to be treated;
a sensor for sensing a type of hair treatment attachment connected to the intermediate attachment; and
an imaging sensor for capturing an image of the hair to be treated.

9. The haircare appliance as claimed in claim 1, wherein the haircare appliance comprises a user feedback module configured to provide feedback to a user based on an output of the sensor module.

10. The haircare appliance as claimed in claim 9, wherein the user feedback module is located in the intermediate attachment.

11. The haircare appliance as claimed in claim 9, wherein the intermediate attachment comprises a feedback controller for controlling the user feedback module based on the output of the sensor module, the user feedback module is located in the main body, and the feedback controller is configured to communicate control instructions to the user feedback module.

12. The haircare appliance as claimed in claim 9, wherein the user feedback module comprises at least one of a haptic feedback module, a visual feedback module, and an aural feedback module.

13. The haircare appliance as claimed in claim 1, wherein the main body comprises a first inductive coil, and the intermediate attachment comprises a second inductive coil for inductively coupling to the first inductive coil.

14. The haircare appliance as claimed in claim 1, wherein the intermediate attachment comprises a power source configured to power the sensor module.

15. The haircare appliance as claimed in claim 1, wherein the intermediate attachment comprises at least one of:
a locking feature for inhibiting rotation of the hair treatment attachment relative to the intermediate attachment;
a flow deflector for deflecting the airflow in use; and
a throttle for throttling the airflow in use.

16. The haircare appliance as claimed in claim 1, wherein the sensor module is located outside of the airflow flowing through the intermediate attachment in use.

17. The haircare appliance as claimed in claim 1, wherein the inlet is opposite the outlet, and the intermediate attachment is configured such that the airflow follows a linear path from the inlet to the outlet in use.

18. The haircare appliance as claimed in claim 1, wherein:
the intermediate attachment has a length measured between the inlet and the outlet; and
the length is less than at least one of a maximum dimension of the main body in a direction parallel to the length when the main body is connected to the intermediate attachment and a maximum dimension of the hair treatment attachment in a direction parallel to the length when the hair treatment attachment is connected to the intermediate attachment.

19. The haircare appliance as claimed in claim 1, wherein the haircare appliance comprises a further hair treatment attachment different to the hair treatment attachment, and the hair treatment attachment and the further hair treatment attachment are interchangeably connectable to the intermediate attachment.

20. An intermediate attachment for a haircare appliance, the intermediate attachment comprising:
a first connection portion for detachably connecting to a main body of the haircare appliance;
a second connection portion for detachably connecting to a hair treatment attachment;
an inlet for receiving an airflow from the main body;
an outlet for emitting the airflow into the hair treatment attachment such that the airflow is emitted from the hair treatment attachment towards hair to be treated in use; and
a sensor module for sensing a property of at least one of the hair to be treated and the hair treatment attachment.
